(19) United States
(12) Patent Application Publication
(10) Pub. No.: US 2009/0209926 A1
(43) Pub. Date: Aug. 20, 2009

(54) UROSTOMY POUCH ADAPTER

(76) Inventor: Sandra Cochran, Chalfont, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/365,948

(22) Filed: Feb. 5, 2009

Related U.S. Application Data
(60) Provisional application No. 61/028,656, filed on Feb. 14, 2008.

(51) Int. Cl. A61F 5/445

(52) U.S. Cl. 604/332

(57) ABSTRACT

The present invention relates generally to a urostomy pouch adapter for attaching a urostomy pouch to a night bag and more particularly, but not exclusively, to a kit comprising a plurality of pouch connectors for attachment to a variety of urostomy pouches and having a night bag connector for providing connection between the pouch connectors and the night bag.

### RELATED APPLICATIONS

This application claims the benefit of priority of U.S. Provisional Application No. 61/028,656, filed on Feb. 14, 2008, the entire contents of which application are incorporated herein by reference.

### FIELD OF THE INVENTION

The present invention relates generally to a urostomy pouch adapter for attaching a urostomy pouch to a night bag and more particularly, but not exclusively, to a kit comprising a plurality of pouch connectors for attachment to a variety of urostomy pouches and having a night bag connector for providing connection between the pouch connectors and the night bag.

### BACKGROUND OF THE INVENTION

Individuals who have bladder cancer typically have a cystectomy (bladder removal) requiring the need for a urostomy pouch which has a compact size that can be conveniently worn during the day so that fluid may be collected as it drains from the stoma. However, while sleeping, a relatively larger volume of fluid produced during the night needs to be collected which can exceed the capacity of a urostomy pouch. One existing solution is to connect an overnight bag via tubing to an outlet port of the urostomy pouch. Despite the fact that urostomy pouches, night bags, and various connectors and tubing exist to attach urostomy pouches to night bags, there is no universal configuration for urostomy pouches. Rather, various makes and models of urostomy pouches exist each of which require their own specialized adapter for connecting the pouch to a night bag. The accompanying adapters, therefore, typically can be used only with a single type of pouch. Consequently, it would be an improvement in the state-of-the-art to provide an adapter kit that can be utilized with a variety of makes and models of urostomy pouches.

### SUMMARY OF THE INVENTION

The present invention provides a urostomy pouch adapter for attaching a urostomy pouch having an exit port to a night bag having an inlet port. The adapter may include a pouch connector having an inlet end and a threaded outlet end. The inlet end may be configured for attachment to the exit port of the urostomy pouch to provide fluid communication between the pouch connector and the interior of the urostomy pouch. The adapter may also include a night bag connector having a threaded inlet end configured for threaded engagement with the outlet end of the pouch connector to provide fluid communication between the night bag connector and the pouch connector. The night bag connector may also have an outlet end adapted to be fluidically connected (directly or indirectly) to the inlet port of the night bag to provide fluid communication between the night bag connector and the interior of the night bag. For example, the outlet end of the night bag connector may be connected to the inlet end of the night bag by a section of tubing.

In another of its aspects, the present invention provides a urostomy pouch adapter kit for attaching a variety of urostomy pouches of differing configuration to a night bag having an inlet port. The kit may include a plurality of pouch connectors each having an outlet end of the same configuration and an inlet end of differing configuration. The inlet end of each pouch connector may be configured for attachment to an exit port of a respective urostomy pouch to provide fluid communication between the pouch connector and the interior of the urostomy pouch. Providing a plurality of pouch connectors having differing inlet end configurations allows the user to utilize a variety of urostomy pouches of differing makes and models. The kit may also include a universal night bag connector having an inlet end configured for engagement with the outlet end of the pouch connectors to provide fluid communication between the night bag connector and the pouch connector. In this regard, the outlet ends of the pouch connectors may have a common configuration so that a single universal night bag connector may be provided for connection to the pouch connectors. The night bag connector may have an outlet end adapted to be fluidically connected (directly or indirectly, e.g., via a section of tubing) to the inlet port of the night bag to provide fluid communication between the night bag connector and the interior of the night bag.

The present invention overcomes difficulties that are presently encountered by many users of urostomy pouches and night bags. For instance, many users of urostomy pouches and night bags suffer from one or more of weakness, carpal tunnel, arthritis, neuropathy, stroke, amputation of any fingers and/or hand, MS disorder, and increased aged. Due to the decrease function of the hands and/or fingers or pain, existing products can be difficult to use, not to mention the possibility of destruction of the appliance with removal, due to the fragile construction of existing adapters.

### BRIEF DESCRIPTION OF THE DRAWINGS

The foregoing summary and the following detailed description of the preferred embodiments of the present invention will be best understood when read in conjunction with the appended drawings, in which:

FIG. 1 schematically illustrates a exemplary urostomy pouch adapter and kit in accordance with the present invention including two pouch connectors and one night bag connector, along with two urostomy pouches and a night bag that may be fluidically connected to one another by the adapter and kit.

### DETAILED DESCRIPTION OF THE INVENTION

Referring now to the figures, wherein like elements are numbered alike throughout, the present invention relates to devices for attaching a urostomy bag 10, 20 to a night bag 80. In particular, the present invention may provide a kit 60 of pouch connectors 30, 40 having an inlet end 32, 42 for attachment to a respective urostomy pouch 10, 20. Providing a variety of pouch connectors 30,40 having differing inlet end configurations permits a variety of different urostomy pouches 10, 20 to be utilized with the kit 60. To reduce the number of parts required in the kit, the outlet end 34, 44 of each pouch connector 30, 40 may have an identical configuration for connection to a single night bag connector 50. Thus, a single, universal night bag connector 50 may be provided having an inlet end 52 for connection to the outlet end 34, 44 of either of the pouch connectors 30,40. The night bag connector 50 may in turn have an outlet end 54 that may be connected to a tube 70 which in turn is connected to an inlet port 82 of the night bag 80, thus providing fluid communication between the night bag 80 and the urostomy pouch 10, 20.

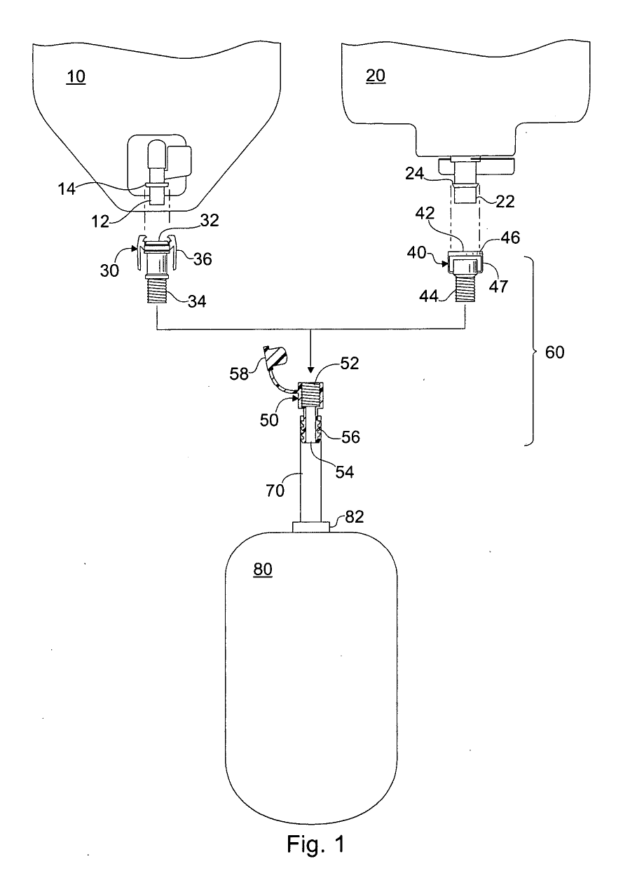

Turning now to FIG. 1 in more detail, the present invention provides a kit 60 of adapters that permits a variety of differing makes and models of existing urostomy bags 10, 20 to be connected to a night bag 80 with a minimum of intermediate parts. Currently, many different companies provide their own proprietary urostomy bags 10, 20 each of which can include an exit port 12, 22 that has a unique configuration that precludes the possibility of a single pouch connector being suitable for connection to each exit port 12, 22. For example, as illustrated in FIG. 1, an exit port 12 of the first urostomy pouch 10 may have a smaller diameter than the exit port 22 of the second urostomy pouch 20, preventing a single pouch connector from being usable with each of the first and second urostomy pouches 10, 20. Consequently, the present invention can provide a plurality of pouch connectors 30, 40, i.e., one connector for each make of pouch with which the kit 60 is to be used. In this regard, each pouch connector 30, 40 may have an inlet end 32, 42 that is configured for connection to a respective model of urostomy pouch 10, 20. For instance, an exemplary first pouch connector 30 in accordance with the present invention may include one or more clips 36 disposed at the inlet end 32 for engagement with a collar 14 disposed at the exit port 12 of the urostomy pouch 10. The clips 36 may be monolithically formed as part of the pouch connector 30 and may be sufficiently flexible and resilient so that the clips 36 may ride up over the collar 14 in snap into place to secure the pouch connector 30 in fluid communication with the exit port 12 of the urostomy pouch 10.

Similarly, an exemplary second pouch connector 40 in accordance with the present invention may include a deformable hoop 46 that is supported at the inlet end 42 of the pouch connector 40 by struts 47. The deformable hoop 46 and struts 47 may be monolithically formed as part of the pouch connector 40. The hoop 46 may be sufficiently flexible so that the hoop 46 may be deformed to assume a shape that allows the hoop 46 to pass over a collar 24 at the output port 22 of the urostomy pouch 20. At the same time, the hoop 46 may be sufficiently resilient so that it can return to an original shape after the deformation so that the hoop 46 cooperates with the collar 24 to secure the pouch connector 40 in fluid communication with the exit port 22 of the urostomy pouch 20.

While a separate pouch connector 30, 40 may be required for each type of pouch 10, 20, by providing an outlet end 34, 44 of each pouch connector 30, 40 with a common configuration, it is possible to utilize a single, universal night bag connector 50 for attachment to the outlet end 34, 44 of the pouch connectors 30, 40. For example, the outlet end 34, 44 of the pouch connectors 30, 40 may be externally threaded and the inlet end 52 of the night bag connector 50 may be internally threaded to receive the threaded outlet ends 34, 44 of the pouch connectors 30, 40 to provide for male-female attachment of either one of the pouch connectors 30, 40 to the night bag connector 50. The night bag connector 50 may optionally include a cap 58 configured to seal the inlet end 52 of the night bag connector 50 when not in use. Likewise, the pouch connectors 30, 40 may optionally include a cap (not shown) to seal the pouch connectors 30, 40 when not in use. Taken together, a single pouch connector 30 or 40 along with the night bag connector 50 comprises an adapter in accordance with the present invention.

To permit fluid communication between the night bag connector 50 and the night bag 80, the outlet end 54 of the night bag connector 50 may be configured for direct or indirect connection with an inlet port 82 of the night bag 80. For example, the outlet end 54 of the night bag connector 50 may include barbs 56 to permit a tube 70 to be attached by pressfit to the outlet end 54 of the night bag connector 50. The opposing end of the tube 70 may similarly be connected to an inlet port 82 of the night bag 80 to effect fluid communication between the night bag 80 and night bag connector 50, and, thus, fluid communication between the night bag 80 and a selected urostomy pouch 10, 20. The tube 70 may be provided as part of the night bag 80, as is presently done with many existing commercial night bags 80. In such a case, the tube 70 supplied with the night bag 80 may include a connector (not shown) which may be removed to permit direct connection between the night bag connector 50 and the tube 70 as illustrated in FIG. 1. Alternatively, an additional length of tubing may be provided to attach the night bag connector 50 to a connector that is already supplied as part of the tube 70 as found in existing commercial night bags 80.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A urostomy pouch adapter for attaching a urostomy pouch having an exit port to a night bag having an inlet port, comprising:
a pouch connector having an inlet end and a threaded outlet end, the inlet end configured for attachment to the exit port of the urostomy pouch to provide fluid communication between the pouch connector and the interior of the urostomy pouch; and
a night bag connector having a threaded inlet end configured for threaded engagement with the outlet end of the pouch connector to provide fluid communication between the night bag connector and the pouch connector, the night bag connector having an outlet end adapted to be fluidically connected to the inlet port of the night bag to provide fluid communication between the night bag connector and the interior of the night bag.

2. A urostomy pouch adapter kit for attaching a variety of urostomy pouches of differing configuration to a night bag having an inlet port, comprising:
a plurality of pouch connectors each having an outlet end and each having an inlet end of differing configuration, the inlet end of the pouch connectors configured for attachment to an exit port of a respective urostomy pouch to provide fluid communication between the pouch connector and the interior of the urostomy pouch; and
a universal night bag connector having an inlet end configured for engagement with the outlet end of the pouch connectors to provide fluid communication between the night bag connector and the pouch connector, the night bag connector having an outlet end adapted to be fluidically connected to the inlet port of the night bag to provide fluid communication between the night bag connector and the interior of the night bag.
